(19) United States
(12) Patent Application Publication
(10) Pub. No.: US 2003/0191088 A1
(43) Pub. Date: Oct. 9, 2003

(54) Novel therapeutic agents interfering in neutrophil migration

(76) Inventor: Ilja Mohandas Hoepelman, Utrecht (NL)

Correspondence Address: TRASK BRITT, P.O. BOX 2550, SALT LAKE CITY, UT, 84110, US

(21) Appl. No.: 10/410,077

(22) Filed: Apr. 9, 2003

Related U.S. Application Data
(63) Continuation of application No. 09/680,671, filed on Oct. 6, 2000, now abandoned.
Application No. 09/680,671 is a continuation of application No. PCT/NL99/00211, filed on Apr. 8, 1999.

(30) Foreign Application Priority Data
Apr. 9, 1998 (EP) 98201154.6

(51) Int. Cl. A61K 31/715; C08B 37/00

(52) U.S. Cl. 514/54; 536/123

(57) ABSTRACT

The present invention provides methods and means to influence neutrophil migration which occurs in a number of serious illnesses, such as acute trauma and inflammatory diseases. In bacterial meningitis for instance a high influx of neutrophils in the brain leads to an unfavorable prognosis. Typically IL-8 is involved in the chemoattraction of neutrophils. According to the present invention, glucoronoxylmannan or a functional equivalent thereof is used to interfere with neutrophil migration. This constitutes a first medical use of this group of substances.

### CROSS-REFERENCE TO RELATED APPLICATION

This application is a continuation of application Ser. No. 09/680,671, filed Oct. 6, 2000, pending, which is a continuation of PCT International Application No. PCT/NL99/00211, filed on Apr. 8, 1999, designating the United States of America.

### TECHNICAL FIELD

The present invention relates to the field of diseases involving the immune system and, in particular, diseases involving neutrophil migration. A number of serious illnesses involve neutrophil transmigration at one stage or another. Such diseases include acute traumas (especially trauma to the brain), infections, ischemic neurological injury and other inflammatory diseases (such as autoimmune diseases).

### BACKGROUND

A very important disease for which no successful drug has been developed so far is bacterial meningitis. Bacterial meningitis is a serious illness, affecting annually in the United States about 3 individuals per 100,000. The case fatality rate is estimated at 25%, and a substantial percentage of the survivors will suffer permanent neurological impairment. Typically, a patient presents with serious clinical symptoms and a high leukocyte count in their cerebrospinal fluid (CSF). Extensive literature evidence suggests that these leukocytes, which are mainly neutrophil leukocytes, play an important adverse role in the pathogenesis of neurological damage.

It has been demonstrated that a high influx of neutrophil leukocytes into the central nervous system (CNS) correlates with an unfavorable prognosis for neurological recovery. Therefore, several attempts have been made to reduce the inflammatory response. So far, the only studies published relating to adjuvant anti-inflammatory treatment in human bacterial meningitis are clinical trials with corticosteroids added to the antibiotic regimen. At least one of the proposed effects of corticosteroids involves the interference of neutrophil migration, although other mechanisms may play a role as well. Results of these clinical trials have been promising though controversial, and only in pediatric cases is the routine use of corticosteroid therapy currently recommended. In adult bacterial meningitis, more evidence of a positive effect of adjuvant corticosteroid treatment is needed.

As previously stated, bacterial meningitis is not the only clinical situation where the influx of neutrophils has been associated with tissue damage. In serious brain injury (brain neurotrauma) and in ischemic events such as cerebrovascular ischemia, spinal cord ischemia, myocardial infarction, intestinal ischemia, pulmonary ischemia, and skeletal muscular ischemia, tissue reperfusion accompanied by neutrophil influx in tissue has repeatedly been correlated to tissue damage. Investigations in the role of neutrophils in noninfectious CNS damage have currently been intensified, since other therapeutic options in this field are very limited.

Experimental animal models have been established to study various anti-inflammatory agents, such as superoxide dismutase, transforming growth factor β, antibodies against adhesion molecules such as CD18 (IB4) and ICAM, caspase inhibitors, polysaccharides and cycloxygenase inhibitors. Many of these strategies aim either directly at the reduction of the neutrophil influx or at the release of toxic neutrophil products into the CNS. Even though the results look promising in animals, the effectiveness of these agents to reduce the migration of neutrophils into the CNS in the human situation remains to be established. Additionally, a considerable concern exists about potential side-effects, such as an adverse reaction to animal proteins (antibodies) and an increased susceptibility to infection through substantial interference with neutrophil function. Therefore, to our knowledge, the agents mentioned have not yet resulted in successful therapeutic tools that can be applied in the human situation. Thus, there is still an urgent need for new therapeutic means, especially in CNS disease.

### DISCLOSURE OF THE INVENTION

The present invention provides a novel means for suppressing neutrophil migration and thus a novel means for preventing damage resulting from neutrophil migration. The invention provides glucuronoxylomannan or a functional equivalent thereof for use as a pharmaceutical.

In particular, the invention provides the use of glucuronoxylomannan or a functional equivalent thereof in the preparation of a medicament for the treatment of a pathological condition or a disease involving neutrophil migration.

In inflammation, soluble factors called chemokines have been identified which attract various subtypes of leukocytes. The chemokine IL-8 is a potent chemoattractant for neutrophils (25). As expected, patients with bacterial meningitis have high levels of IL-8 as well as an increased number of neutrophils in their cerebrospinal fluid (CSF). Patients were recently described who suffered from a fungal meningitis caused by Cryptococcus neoformans, whose CSF typically contains very little leukocytes, had high levels of IL-8 in their CSF {26). Therefore, in cryptococcal meningitis, an agent must be present that interferes with neutrophil migration towards IL-8. Patients with cryptococcal infection have high titers of the fungal capsular polysaccharide glucuronoxylomannan (GXM) in both serum and CSF during infection (27, 28). Cryptococcal polysaccharides have been shown to interfere with neutrophil movement (29, 30, 31, and 32}. Moreover, it has been demonstrated in vitro that GXM also interferes with neutrophil migration (chemotaxis) towards IL-8 if GXM is added directly to the leukocytes (26). It has also demonstrated that a low CSF leukocyte cell count in patients with cryptococcal meningitis shows a significant inverse correlation to a high GXM titer in serum (relative to the GXM titer in CSF) (Lipovsky, N. N., L. van Elden, A. E. Waterkamp, I. Dankert, A.I.M. Hoepelman, “Does the capsule component of the Creptococcus neoformans glucuronoxylomannan impair transendothelial migration of leukocytes in patients with cryptococcal meningitis?”, The Journal of Infectious Diseases, 98 vol. 178 p. 1231-32).

In a pneumococcal meningitis model in rabbits, we have recently indeed shown that intravenous administration of GXM not only delays entry of leukocytes (PMN) into the CSF and brain, but more importantly reduces TNF-α levels in the CSF and also protects the brains of these rabbits against damage in comparison to the control rabbits.

The exact mechanism of how GXM interferes with neutrophil migration in cryptococcosis is not known. GXM consists of an α-1, 3-linked polymannose backbone with O-acetyl substituents and β-linked monomeric branches of xylose and glucuronic acid (72). GXM has been shown to attract neutrophils itself (30, 26). Moreover, GXM has been shown to interfere with molecules that are essential in neutrophil-endothelial interaction, for instance: it can bind to CD18 (which is the β subunit of leucocyte function associated antigen-1, LFA-1) (35), and GXM can induce shedding of neutrophil L-selectin (36), a molecule that is involved in the first steps of endothelial leukocyte transmigration.

GXM is easily purified (37), and, in its purified form, it is poorly immunogenic (38). Moreover, patients with cryptococcal meningitis can display relatively mild symptoms for a prolonged period of time despite detectable levels of GXM (in blood and CSF) (27), probably indicating that GXM by itself is non-toxic. Therefore, GXM is a potent therapeutic agent. Of course, functional equivalents of GXM or the cryptococcal cell wall will be capable of the same interference with IL-8 or otherwise-induced migration of neutrophils. Thus, GXM or a functional equivalent thereof can be applied in the treatment of diseases or pathological conditions involving neutrophil migration. Typically, such diseases include infections, disease characterized by ischemia or other inflammatory diseases. GXM will be especially useful in diseases which involve IL-8 regulation. Examples thereof are brain inflammatory diseases such as meningitis (in particular bacterial meningitis), and neurotrauma or the protection of the brain against toxic medication administered systemically (e.g., anti-cancer drugs).

Other important applications will include the treatment of pathological conditions such as brain injury or ischemic events, as described herein. In a further embodiment, the invention provides a pharmaceutical composition comprising glucuronoxylomannan or a functional equivalent thereof together with a suitable means for administration. Typically, such a composition will be given systemically. The dose of GXM necessary to prevent neutrophil migration may vary, but will generally be between 5 mg/kg and 250 mg/kg, and preferably between 25 mg/kg and 150 mg/kg.

The compositions may, of course, contain other drugs for the treatment of diseases involving neutrophil migration, such as anti-inflammatory drugs, corticosteroids or an antibiotic agent.

The invention will be explained in more detail in the following experimental part.

Isolation of GXM.

C. neoformans is autoclaved after growth in a chemically defined medium for 5 days. Polysaccharide is precipitated with calcium acetate and ethanol. After being dissolved in NaCl and undergoing brief ultrasonic irradiation, the polysaccharide is precipitated by differential complexation with hexadecyltrimethylammonium bromide. Purified GXM is precipitated by ethanol, dissolved, ultrasonically irradiated for 2 h, centrifuged, dialyzed, and finally recovered by lyophilization. Samples are analyzed through various methods to prove that none of the purified polysaccharides contain constituents other than those known to occur in GXM (37).

Animal Toxicity.

In experiments, it was investigated whether GXM is toxic in healthy animals.

It is demonstrated that healthy rabbits tolerated intravenous administration of GXM in doses ranging from 0.5 to 50 mg. Moreover, in the pneumococcal meningitis rabbit model, i.v. administration of GXM also proved to be non-toxic and well tolerated. No demonstrable brain toxicity was found, while in CSF, GXM was detectable in these rabbits.

References

1. Schlech WFI, Ward J I, Band J D, et al. Bacterial meningitis in the United States, 1978 through 1981. The national bacterial meningitis surveillance study. JAMA 1985; 253:1749-54.

2. Durand M L, Calderwood S B, Weber D J, et al. Acute bacterial meningitis in adults: a review of 493 episodes. N Engl J Med 1993; 328:21-8.

3. Pfister H W, Fontana A, Tauber M G, Tomasz A, Scheld W M. Mechanisms of brain injury in bacterial meningitis: workshop summary. Clin Infect Dis 1994; 19:463-79.

4. Spellerberg B, Tuomanen E I. The pathophysiology of pneumococcal meningitis. Ann Med 1994; 26:411-8.

5. Tuomanen E I, Saukkonen K, Sande S, Cioffe C, Wright S D. Reduction of inflammation, tissue damage, and mortality in bacterial meningitis in rabbits treated with monoclonal antibodies against adhesion-promoting receptors of leukocytes. J Exp Med 1989; 170:959-69.

6. Saez Llorens X, Jafari H S, Severien C, et al. Enhanced attenuation of meningeal inflammation and brain edema by concomitant administration of anti-CD 18 monoclonal antibodies and dexamethasone in experimental Haemophilus meningitis. J Clin Invest 1991; 88:2003-11.

7. Cronstein B N, Kimmel S C, Levin R I, Martiniuk F., Weissmann G. A mechanism for the antiinflammatory effects of corticosteroids: the glucocorticoid receptor regulates leukocyte adhesion to endothelial cells and expression of endothelial-leukocyte adhesion molecule 1 and intercellular adhesion molecule 1. Proc Natl Acad Sci USA 1992; 89:9991-5.

8. Quagliarello V J, Scheld W M. Treatment of bacterial meningitis. N Engl J Med 1997; 336:708-16.

9. Hartl R, Medary M B, Ruge M, Arfors K E, Ghajar J. Early white blood cell dynamics after traumatic brain injury: effects on the cerebral microcirculation. J Cereb Blood Flow Metab 1997; 17:1210-20.

10. Hallenbeck J M. Cytokines, macrophages, and leukocytes in brain ischemia. Neurology 1997; 49:S5-9.

11. Winquist R J, Kerr S. Cerebral ischemia-reperfusion injury and adhesion. Neurology 1997; 49:S23-6.

12. Yanaka K, Camarata P J, Spellman S R, McCarthy J B, Furcht L T, Low W C. Antagonism of leukocyte adherence by synthetic fibronectin peptide V in a rat model of transient focal cerebral ischemia. Neurosurgery 1997; 40:557-63.

13. Korthuis R J, Anderson D C, Granger D N. Role of neutrophil-endothelial cell adhesion in inflammatory disorders. J Crit Care 1994; 9:47-71.

14. Tanaka M, Brooks S E, Richard V J, et al. Effect of anti-CD18 antibody on myocardial neutrophil accumulation and infarct size after ischemia and reperfusion in dogs. Circulation 1993; 87:526-35.

15. Schoenberg M H, Poch B, Younes M, et al. Involvement of neutrophils in postischemic damage to the small intestine. Gut 1991; 32:905-12.

16. Kubes P, Hunter J, Granger D N. Ischemia/reperfusion-induced feline intestinal dysfunction: importance of granulocyte recruitment. Gastroenterology 1992; 103:807-12.

17. Sekido N, Mukaida N, Harada A, Nakanishi I, Watanabe Y, Matsushima K. Prevention of lung reperfusion injury in rabbits by a monoclonal antibody against interleukin-8. Nature 1993; 365:654-7.

18. Petrasek P F, Liauw S, Romaschin A D, Walker P M. Salvage of postischemic skeletal muscle by monoclonal antibody blockade of neutrophil adhesion molecule CD18. J Surg Res 1994; 56:5-12.

19. Clark W M, Zivin J A. Antileukocyte adhesion therapy: preclinical trials and combination therapy. Neurology 1997; 49:S32-8.

20. Tuomanen E. Adjunctive therapy of experimental meningitis: agents other than steroids. Antibiot Chemother 1992; 45:184-91.

21. Ley K, Cerrito M, Arfors K E. Sulfated polysaccharides inhibit leukocyte rolling in rabbit mesentery venules. Am J Physiol 1991; 260:H 1667-73.

22. Ley K, Linncmann G, Meinen M, Stoolman L, Gaehtgens P. Fucoidin, but not yeast polyphosphomannan PPME, inhibits leukocyte rolling in venules of the rat mesentery. Blood 1993; 81:177-85.

23. Adams D H, Lloyd A R. Chemokines: leukocyte recruitment and activation cytokines. Lancet 1997; 349:490-5.

24. Taub D D. Chemokine-leukocyte interactions: the voodoo that they do so well. Cytokine Growth Factor Reviews 1996; 7: 355-76.

25. Bell M D, Taub D D, Perry V H. Overriding the brain's intrinsic resistance to leukocyte recruitment with intraparenchymal injections of recombinant chemokines. Neuroscience 1996; 74:283-92.

26. Chaka W S, Heyderman R, Gangaidzo I, et al. Cytokine profiles in CSF of HIV-infected patients with cryptococcal meningitis: no leukocytosis despite high IL-8 levels. J Infect Dis 1997; 176:1633-6.

27. Mitchell T G, Perfect J R. Cryptococcosis in the era of AIDS-100 years after the discovery of Cryptococcus neoformans. Clinical Microbiology Reviews 1995; 8:515-48.

28. Eng RHK, Bishburg E, Smith S M. Cryptococcal infections in patients with acquired immune deficiency syndrome. Am J Med 1986; 81:19-23.

29. Drouhet E, Segretain G. Inhibition de la migration leucocytaire in vitro par un polyoside capsulaire de Torulopsis (Cryptococcus) neoformans. Ann Inst Pasteur 1951; 81:674-6.

30. Dong Z M, Murphy J W. Effects of two varieties of Cryptococcus neoformans cells and culture filtrate antigens on neutrophil locomotion. Infect Immun 1995; 63:2632-44.

31. Dong Z M, Murphy J W. Intravascular cryptococcal culture filtrate (CneF) and its major component, glucuronoxylomannan, are potent inhibitors of leukocyte accumulation. Infect Immun 1995; 63:770-8.

32. Dong Z M, Murphy J W. Mobility of human neutrophils in response to Cryptococcus neoformans cells, culture filtrate antigen, and individual components of the antigen. Infect Immun 1993; 61:5067-77.

33. Lipovsky M M, Gekker G, Hu S, Ehrlich L C, Hoepelman A I M, Peterson P K. Cryptococcal glucuronoxylomannan induces interleukin (IL)-8 production by human microglia but inhibits neutrophil migration towards IL-8. J Infect Dis 1998; 177:260-3.

34. Cherniak R, Sundstrom J B. Polysaccharide antigens of the capsule of Cryptococcus neoformans. Infect Immun 1994; 62:1507-12.

35. Dong Z M, Murphy J W. Cryptococcal polysaccharides bind to CD18 on human neutrophils. Infect Immun 1997; 65:557-63.

36. Dong Z M, Murphy J W. Cryptococcal polysaccharides induce L-selectin shedding and tumor necrosis factor receptor loss from the surface of human neutrophils. J Clin Invest 1996; 97:689-98.

37. Cherniak R, Morris L C, Anderson B C, Meyer S A. Facilitated isolation, purification, and analysis of glucuronoxylomannan of Cryptococcus neoformans. Infect Immun 1991; 59:59-64.

38. Eckert T F, Kozel T R. Production and characterization of monoclonal antibodies specific for Cryptococcus neoformans capsular polysaccharide. Infect Immun 1987; 55:1895-9.

39. Burroughs M H, Tsenova Berkova L, Sokol K, Ossig J, Tuomanen E, Kaplan G. Effect of thalidomide on the inflammatory response in cerebrospinal fluid in experimental bacterial meningitis. Microb Pathog 1995; 19:245-55.

What is claimed is:

1. Glucuronoxylomannan or a functional equivalent thereof for use as a pharmaceutical.

2. Use of glucuronoxylomannan in the preparation of a medicament for the treatment of a pathological condition or a disease involving neutrofil migration.

3. Use according to claim 2 whereby the disease is an infection or an inflammatory disease.

4. Use according to claim 3, whereby the inflammatory disease involves IL-8 regulation.

5. Use according to anyone of claims 2-4, whereby the inflammatory disease is meningitis, in particular bacterial meningitis.

6. Use according to claim 2 whereby the pathological condition is serious brain injury or an ischemic event.

7. Use according to claim 6 whereby the ischemic event is a myocardial infarct, cerebrovascular ischemia, intestinal ischemia, pulmonary ischemia or skeletal muscular ischemia.

8. A pharmaceutical composition comprising gucuronoxylmannan or a functional equivalent thereof together with a suitable means for administration.

9. A pharmaceutical composition according to claim 8, further comprising a further anti-inflammatory drug.

10. A pharmaceutical composition according to claim 8 or 9, further comprising a corticosteroid.

11. A pharmaceutical composition according to any one of claims 9-10, further comprising an antibiotic agent.

12. A method for preventing the migration of neutrophils towards IL-8, comprising providing said neutrophils with a composition comprising glucuronoxylomannan.

13. The method according to claim 1, wherein the composition comprising glucuronoxylomannan is provided to a subject.

14. The method according to claim 2, wherein the composition comprises glucuronoxylomannan together with a suitable means for administration.

15. The method according to claim 2 or 3, wherein the dose of glucuronoxylomannan to the subject is between 5 mg/kg and 250 mg/kg.

16. The method according to claim 4, wherein the dose of glucuronoxylomannan to the subject is between 25 mg/kg and 150 mg/kg.
